(19) United States
(12) Patent Application Publication
(10) Pub. No.: US 2017/0367930 A1
(43) Pub. Date: Dec. 28, 2017

(54) Emergency Medication Dose Device to Provide Error-Free Pediatric Medication Doses.

(71) Applicants: Elizabeth Gompf, San Diego, CA (US); Charles Gompf, San Diego, CA (US)

(72) Inventors: Elizabeth Gompf, San Diego, CA (US); Charles Gompf, San Diego, CA (US)

(21) Appl. No.: 15/628,829

(22) Filed: Jun. 21, 2017

Related U.S. Application Data
(60) Provisional application No. 62/355,445, filed on Jun. 28, 2016.

(51) Int. Cl. A61J 1/03; A61B 90/92; A61B 50/30

(52) U.S. Cl. CPC A61J 1/03; A61B 90/92; A61B 50/30; A61B 2050/3008; A61J 2205/20

(57) ABSTRACT

This invention provides a means by which correct dosages of emergency medication can be provided to children under emergency situations. A color- and size-coded set of cylinders is provided where the colors match the color codes of the Broselow® Pediatric Emergency Tape, a common method by which the proper dosage for child is estimated based on the child's height. Each type of medication in the emergency kit has its own color-coded set of cylinders, such that once emergency medical personnel measure the child and determine which medication to use, all they need to do is grab the correct cylinder based on the color (which has already been determined from the Broselow® measurement). The invention allows emergency medical personnel to safely, efficiently, quickly, and accurately deliver the correct dosage of one or more medications to a child under emergency conditions.

### CROSS REFERENCE TO RELATED APPLICATIONS

This utility patent application claims priority to U.S. Provisional Application No. 62/355,445, filed Jun. 28, 2016, a copy of which is attached to this application and the contents of which are incorporated by reference.

### STATEMENT REGARDING FEDERALLY SPONSORED RESEARCH OR DEVELOPMENT

This invention was not federally sponsored.

### BACKGROUND OF THE INVENTION

### Field of the Invention

This invention relates to the general field of emergency medication and the correct application of the same, and more specifically toward a device that contains color-coded cylinders for a variety of medications which can be appropriately selected based on matching the color with a Broselow® Pediatric Emergency Tape. This invention allows emergency medical personnel to deliver correct dosages of medication for children of different lengths under emergency situations. A color- and size-coded set of cylinders is provided where the colors match the color codes of the Broselow® Pediatric Emergency Tape, a common method by which the proper dosage for child is estimated based on the child's length. Each type of medication in the emergency kit has its own color-coded set of cylinders, such that once emergency medical personnel measure the child and determine which medication to use, all he/she needs to do is grab the correct cylinder based on the color (which has already been determined from the Broselow® measurement).

### Statement of the Problem Solved

Emergency medication for adults is delivered in ampules by the Hospira Abboject™ syringe based upon the size of an average adult. For children, this adult dose is too great for their size. The invention is used to provide a measured dose of emergency medication calibrated for children based upon a range of weights.

The most common way to quickly estimate the weight of a child is the Broselow® measuring tape. The Broselow® tape is a length of material upon which a universal set of color-coded “dosing zones” are laid out, such that a medical worker trying to estimate what the proper dosage is for a particular child can lay Broselow® tape next to the child and see which color-coded “zone” he or she fits in, then determine the proper dosage for that particular child. This length-based dosing is particularly relevant for emergency medical situations. As a contrast, for example, when a child goes to a pediatrician for a check-up, a nurse generally weighs the child before seeing the doctor, so the doctor knows what dosage of a particular medication to give this child based on the known weight of the child. However, under emergency circumstances, there generally are not scales readily available for weighing children.

Measuring the proper dose of medication to give to a child under emergency conditions is extremely challenging. For example, a medical practitioner trying to revive a child with CPR will not be able to focus well on the proper dosage for that child. The child dosage is delivered from an adult emergency ampule, which requires calculations and measurements that must be precise during a critical event. Further complicating the situation, over- or under-dosing a child often has severe, and sometimes fatal repercussions. Indeed, it is estimated that up to 80% of dosages given to children under emergency conditions are incorrect for the size of the child, and some studies state that errors in pediatric dosages are a substantial factor in childhood death. Thus, treating children injured during emergency situations is extremely stressful on the medical responders.

Assessing a child's weight and then drawing the correct amount of an emergency medication before delivering it to a pediatric patient also takes time. Considering that many dosages of emergency medication to children are done under significant time and locational challenges, it is understandable that any device that would both increase the likelihood that the correct dosage is given, and save time, is highly desirable.

### Prior Art

There is considerable prior art that attempts to deliver measured, accurate dosages, but none provide the solution so cleanly provided by this invention. There are inventions that attempt to measure a dose before the dose is inserted into a syringe. However, none of the prior art provides a device that is easy to use and provides a virtually fool-proof method of administering the correct dosage to a child under emergency circumstances. In short, since the introduction of the Broselow® emergency tape in 1985, there have not been significant improvements in the selection of medication dosages to accompany an assessment of a child's weight through the Broselow® tape.

This invention provides a means by which correct dosages of emergency medication can be provided to children under emergency situations. A color- and size-coded set of cylinders is provided where the colors match the color codes of the Broselow® Pediatric Emergency Tape, a common method by which the proper dosage for child is estimated based on the child's length. Each type of medication in the emergency kit has its own color-coded set of cylinders, such that once emergency medical personnel measure the child and determine which medication to use, all he/she needs to do is grab the correct cylinder based on the color (which has already been determined from the Broselow® measurement). The invention also provides additional measures of safety in the each cylinder is labeled with medication and dose information (for example: medication name, concentration, patient weight range, dosage by weight/volume) that can be verified with medication vial and patient height/weight information.

### SUMMARY OF THE INVENTION

The invention disclosed in this patent application has a number of components which, when combined together, provides a solution to the ongoing problem of how emergency medical professionals administer the correct dosage to a child under emergency conditions.

It is a principal object of the invention to provide a color- and size-coded set of cylinders that correspond to the color codes of the Broselow® Pediatric Emergency Tape.

It is another object of the invention to provide an emergency kit that contains a number of different vials of medications commonly used to treat children, where each type of medication has multiple cylinders that are color coded to correspond to the color codes of the Broselow® Pediatric Emergency Tape.

It is a final object of this invention to provide a means by which emergency medical personnel can accurately and quickly administer the correct dosage of a medication to a child.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE FIGURES

The accompanying drawings, which are incorporated in and form a part of this specification, illustrate embodiments of the invention and together with the description, serve to explain the principles of this invention.

FIG. 1 is a front view of the invention illustrating the basic physical structure of the insert.

FIG. 2 is a cross-sectional view of the insert shown in FIG. 1.

FIG. 3 is a top view of the insert shown in FIG. 1.

FIG. 4 is a perspective view of the insert shown in FIG. 1.

FIG. 5 is a cross-sectional view of the insert, syringe, and vial, which are the three basic components, that when used correctly, can provide an error-free dosage to a child.

FIG. 6 is a series of drawings showing cross-sectional depictions showing the insert positioning prior to and after installation into a syringe.

FIG. 7 is a series of drawings showing cross-sectional depictions showing how the insert appears when placed within the syringe and the vial is being attached.

FIG. 8 is a series of cross-sectional views showing the vial being pressed into the syringe, delivering a dose limited by the step or “blocking ledge” in the insert.

FIG. 9 is a cross-sectional view of the vial after it has been depressed as far as it will go, thereby limiting the dosage given to the child.

FIG. 10 is a perspective view of a series of Epinephrine inserts, each color coded to the appropriate “zone” of the Broselow® tape.

FIG. 11 is a front view of how the invention might appear in an EMT's medication kit, with different color-coded inserts for different medications, with each insert color coded to the appropriate “zone” of the Broselow® tape.

FIG. 12 is a front view of how the invention might appear in an EMT's medication kit, with different color-coded inserts for different medications, with each insert color coded to the appropriate “zone” of the Broselow® tape and the type of medication written on each insert.

FIG. 13 is a front view of how the invention might appear in another embodiment of an EMT's medication kit, with different color-coded inserts for different medications, with each insert color coded to the appropriate “zone” of the Broselow® tape and the inserts organized by dosage category.

### DETAILED DESCRIPTION OF THE INVENTION

Many aspects of the invention can be better understood with the references made to the drawings below. The components in the drawings are not necessarily drawn to scale. Instead, emphasis is placed upon clearly illustrating the components of the present invention. Moreover, like reference numerals designate corresponding parts through the several views in the drawings.

By way of a quick summary of the invention, the insert is color-coded to a particular Broselow® child size. Each insert has a blocking ledge in a certain position such that it provides a stopping point beyond which a vial of medication cannot be pushed, thereby limiting the amount of medication that is delivered to a child. As the vial is depressed, medication flows through the needle and is stopped when the outer edge of the vial contacts the blocking ledge, thereby limiting the amount of medication delivered by the syringe. Since the blocking ledges are located at different levels of the insert depending on the correct dosage for a child of that Broselow® color code, all emergency medical personnel have to do is select the proper color and medication from the kit provided.

### REFERENCE NUMBERS USED

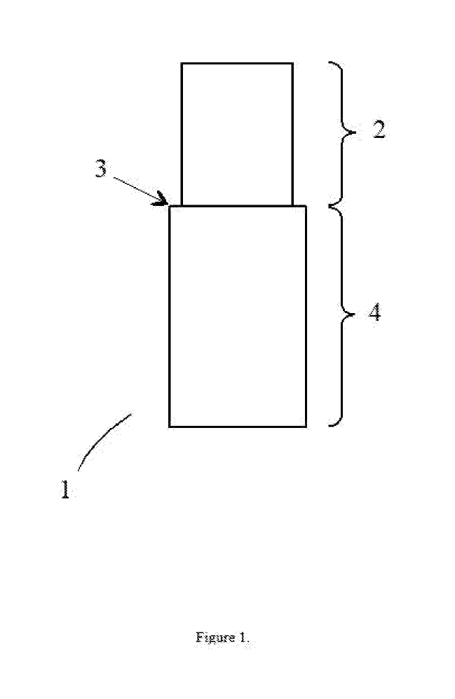

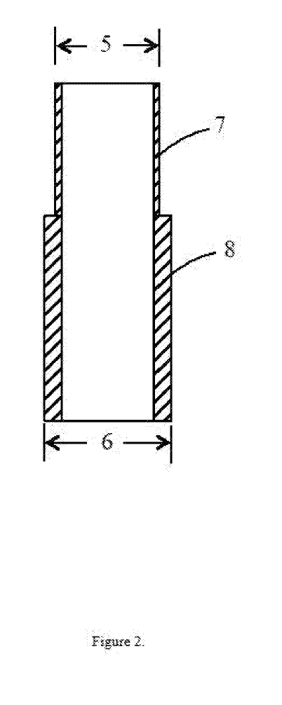

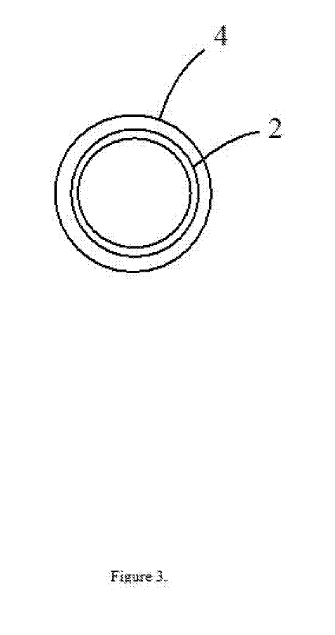

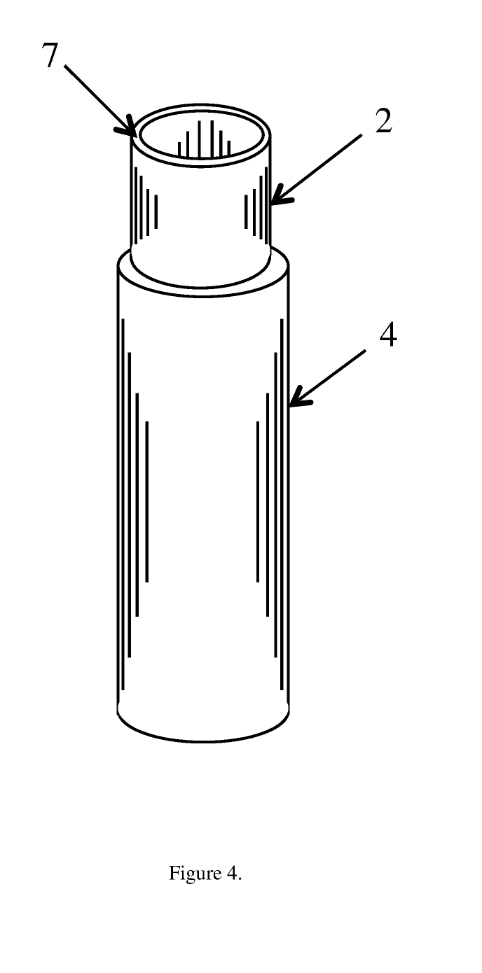

1. Insert Generally
2. Insert Top Portion
3. Blocking ledge
4. Insert Bottom Portion
5. Insert top outer diameter
6. Insert bottom outer diameter
7. Insert Top Wall thickness
8. Insert Bottom Wall thickness
9. Syringe
10. Vial
11. Vial wall width
12. Vial bottom
13. Vial wall
14. Medication
15. Rubber plunger
16. Insert bottom
17. Vial lip
18. Left Blank on purpose
19. Left Blank on purpose
20. Left Blank on purpose
21. Left Blank on purpose
22. Vial internal diameter
23. Left Blank on Purpose
24. Left Blank on purpose
25. Left Blank on purpose
26. Syringe outer chamber width
27. Syringe outer chamber bottom
28. Syringe inner chamber
29. Needle
30. Syringe inner chamber stopper
31. Syringe cavity
FIG. 1 is a front view of the invention illustrating the basic physical structure of the insert. FIG. 2 is a cross-sectional view, FIG. 3 is a top view and FIG. 4 is a perspective view of the insert shown in FIG. 1.

The insert, generally referenced as 1, is a cylindrical container with two sections: an insert top portion 2 which has a narrower outside diameter 5 than the second part, an insert bottom portion 4, which has a larger diameter 6. This is caused by the fact that the insert top wall thickness 7 has a thinner width than the width of the insert bottom wall thickness 8. The increase in outside diameter between the insert top 2 and the insert bottom 4 causes a blocking ledge 3. The insert 1 is placed in a syringe (in a preferred embodiment, the syringe is a Hospira Abboject®), with insert top 2 upright, and a vial of medication is installed by screwing the vial stopper into the syringe over the insert. The vial wall is then pushed down and medication flows through the syringe needle until the vial wall's progress is stopped by the blocking ledge of the insert hitting a vial lip (17 as referenced in other figures). Thus, by adjusting the location of the blocking ledge 3, the volume of medication delivered can be changed from insert to insert, such that the proper dosage is given to a child of that particular size.

The outside diameter of the insert top 2 is small enough to allow a vial wall to slip over it, and the blocking ledge 3 is wide enough to prevent the vial wall from slipping any further down the insert, thereby limiting the amount of medication delivered from the vial through the syringe.

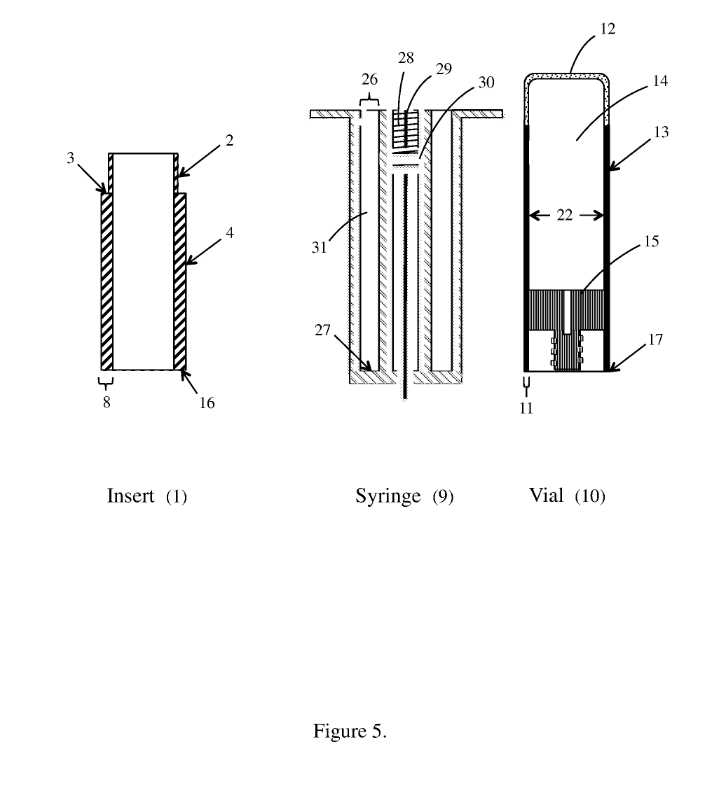

FIG. 5 is a cross-sectional view of the insert, syringe, and vial, which are the three basic components, that when used correctly, can provide an error-free dosage to a child. The syringe 9 is a cylindrical container with a needle 29 at one end and an open top. The insert 1 is slid into the open top and nestled at the bottom of the syringe. The vial 10 is a cylinder with a vial wall 13 with a vial wall width 11, and open top defined by a vial lip 17, a vial bottom 12, where the inner sides of the vial wall define a vial internal diameter 22. Inside the cylinder is a quantity of medication 14, and retaining the medication in the vial is a rubber plunger 15. After insert 1 is placed within the syringe 9, the vial is screwed onto the syringe. When properly installed the syringe needle 29 penetrates the rubber plunger 15 that is used to seal the vial of medication. Note that as the vial is pushed down, the medication 14 will be directed through the needle 29 until the vial lip 17 hits the blocking ledge 3.

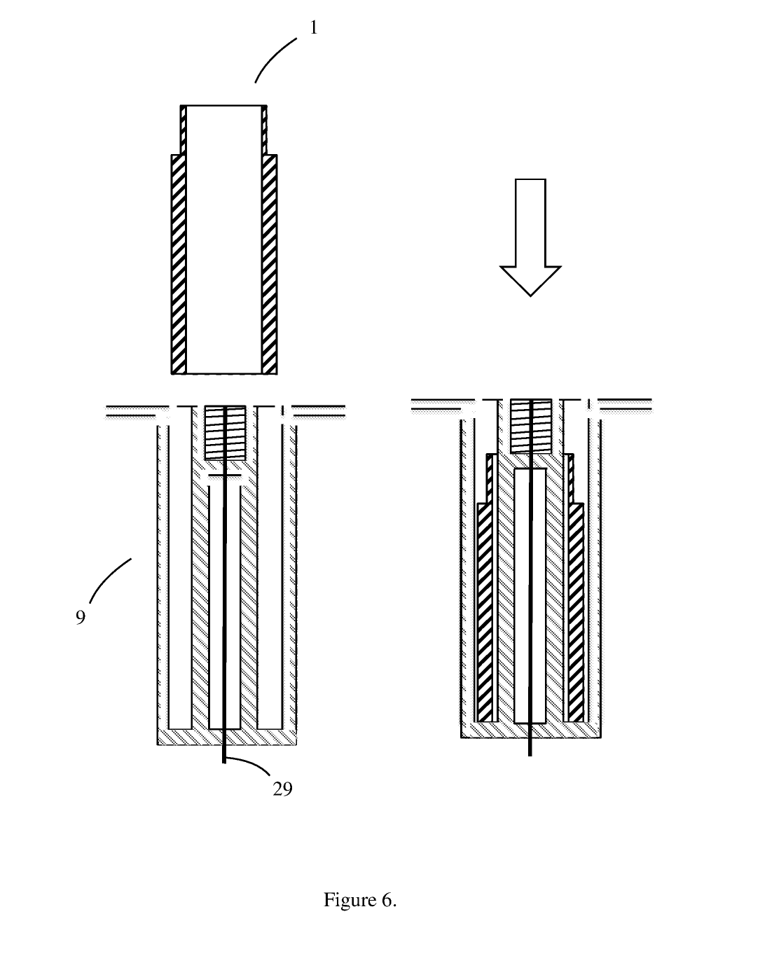

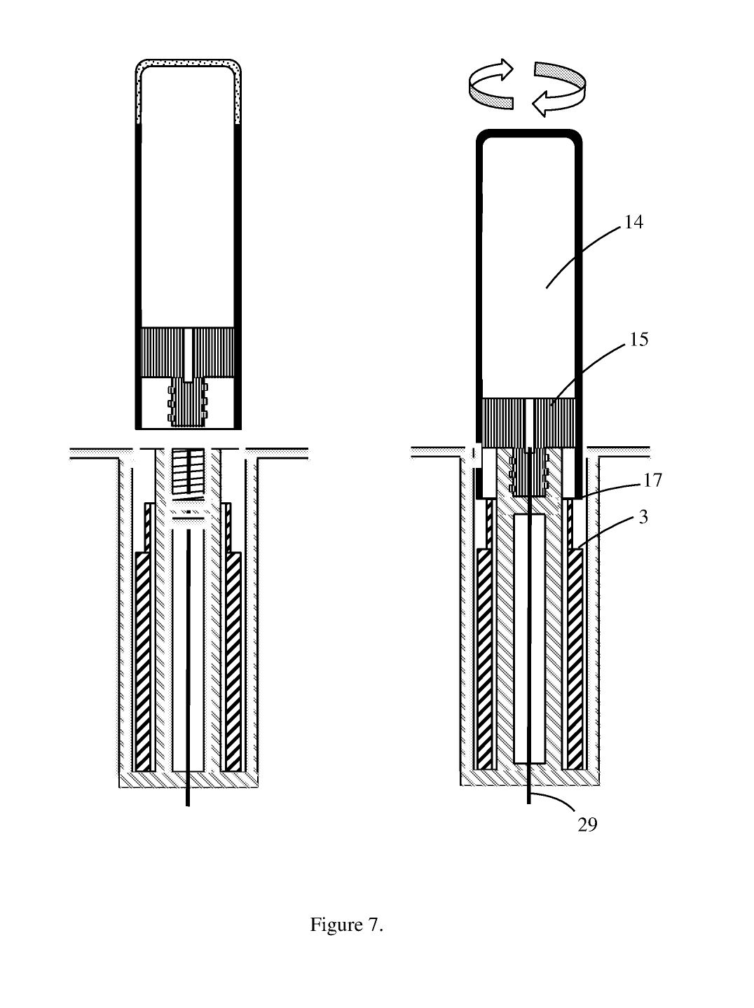

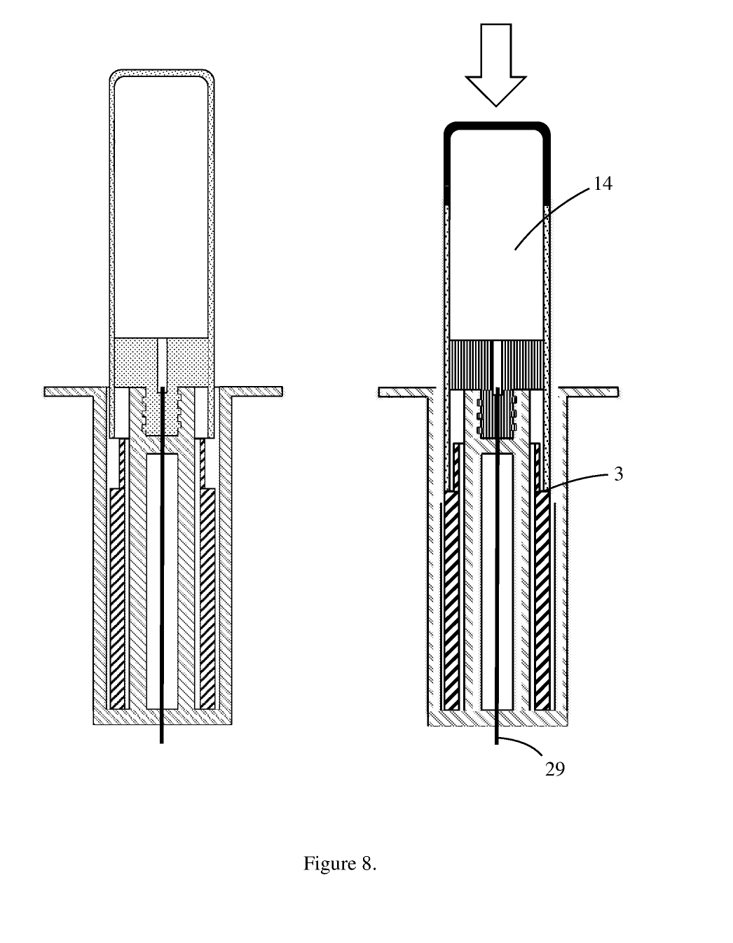

FIG. 6 is a series of drawings showing cross-sectional depictions showing the insert positioning prior to and after installation into a syringe. FIG. 7 is a series of drawings showing cross-sectional depictions showing how the insert appears when placed within the syringe and the vial is being attached. FIG. 8 is a series of cross-sectional views showing the vial bring pressed into the syringe, delivering a dose limited by the step or “blocking ledge” in the insert.

The insert 1 is slid into the syringe cavity 31 of the syringe 9 until the insert bottom 16 hits the syringe outer chamber bottom 27. The syringe outer chamber width 26 is wide enough to accommodate the insert bottom wall thickness 8, such that the insert 1 slides easily into the syringe 9. The vial 10 is then attached to the syringe 9. After the vial is installed on the syringe, the vial bottom 12 is pushed toward the blocking ledge 3, the appropriate amount of medication 14 will flow through the needle 29, until the vial lip 17 contacts the blocking ledge 3. When the vial has been fully compressed to the point where the vial lip 17 has hit the blocking ledge 3, the correct quantity of medication 14 has been properly delivered. The vial 10 is then pressed down. The vial internal diameter 22 is larger than the insert top outer diameter 5, but less than the insert bottom outer diameter 6, such that vial lip 17, cannot be pushed beyond the blocking ledge 3. Thus, a set quantity of medication is injected through the needle 29 in the syringe inner chamber 28, as the rubber plunger 15 in the vial mates to the syringe inner chamber stopper 30, such as the medication 14 in the vial is injected up to the point where the vial lip 17 hits the blocking ledge 3. Because the blocking ledges are located at different heights in different colors of inserts, emergency medical personnel can easily pick the appropriate insert by matching it to the appropriate color code of the Broselow® Pediatric Emergency Tape.

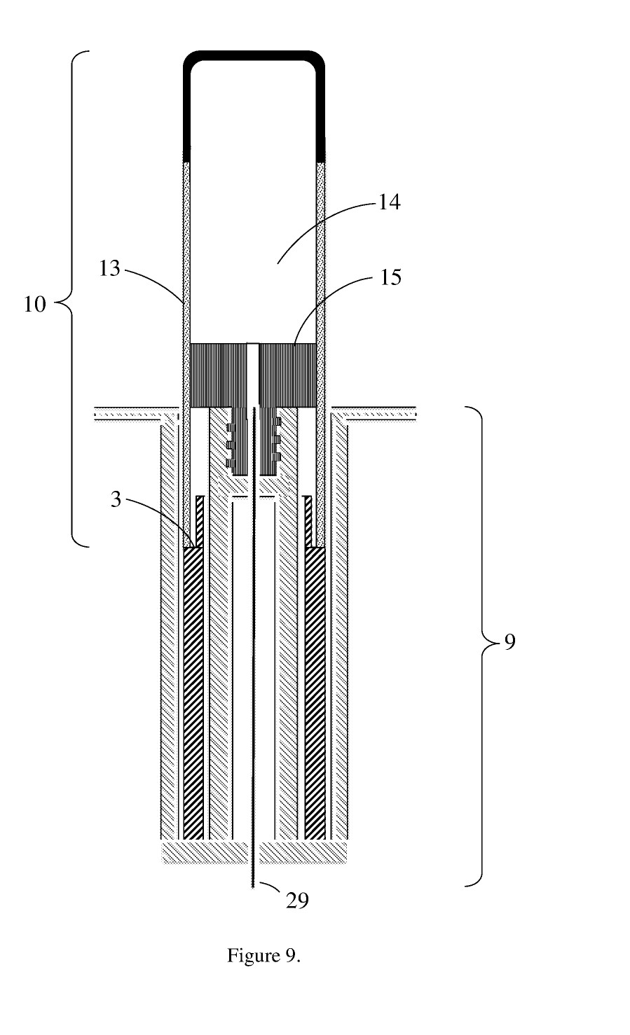

FIG. 9 is a cross-sectional view of the vial after it has been depressed as far as it will go, thereby limiting the dosage given to the child. The vial 10 has been pushed into the syringe 9 as far as it will go based upon the color of insert that was used. The blocking ledge 3 of the insert has stopped the vial wall 13 from entering the syringe 9 further, thereby limiting the amount of medication 14, that was injected into the child. The threaded rubber plunger 15 has directed the medication 14 through the needle 29.

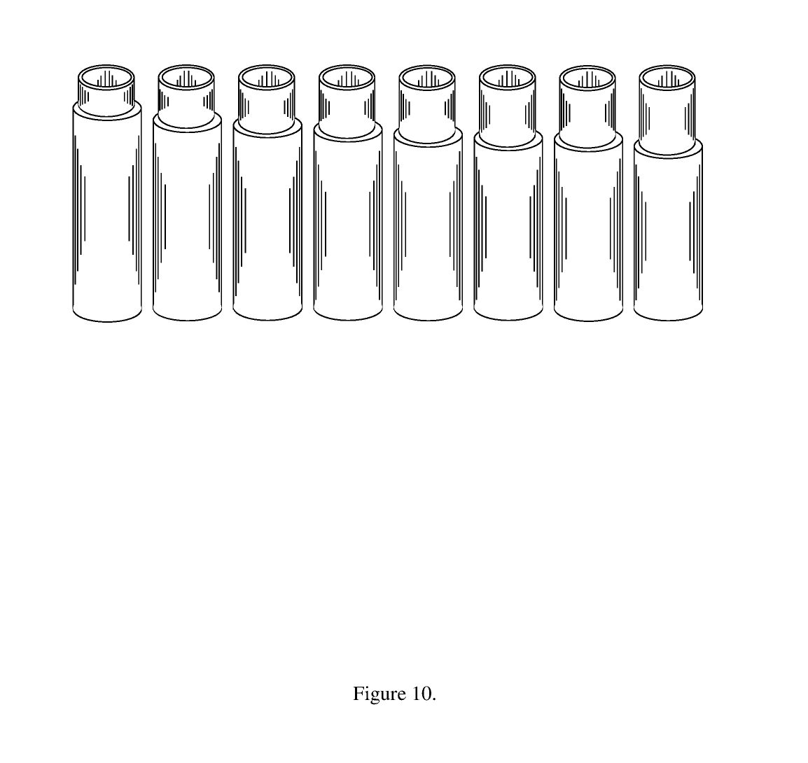

FIG. 10 is a perspective view of a series of Epinephrine inserts, each color coded to the appropriate “zone” of the Broselow® tape. Note how the blocking ledge is a different distance “down” each insert, so that a particular color of insert will allow the proper dosage of a medication to a child whose height corresponds to the particular Broselow® color “zone”.

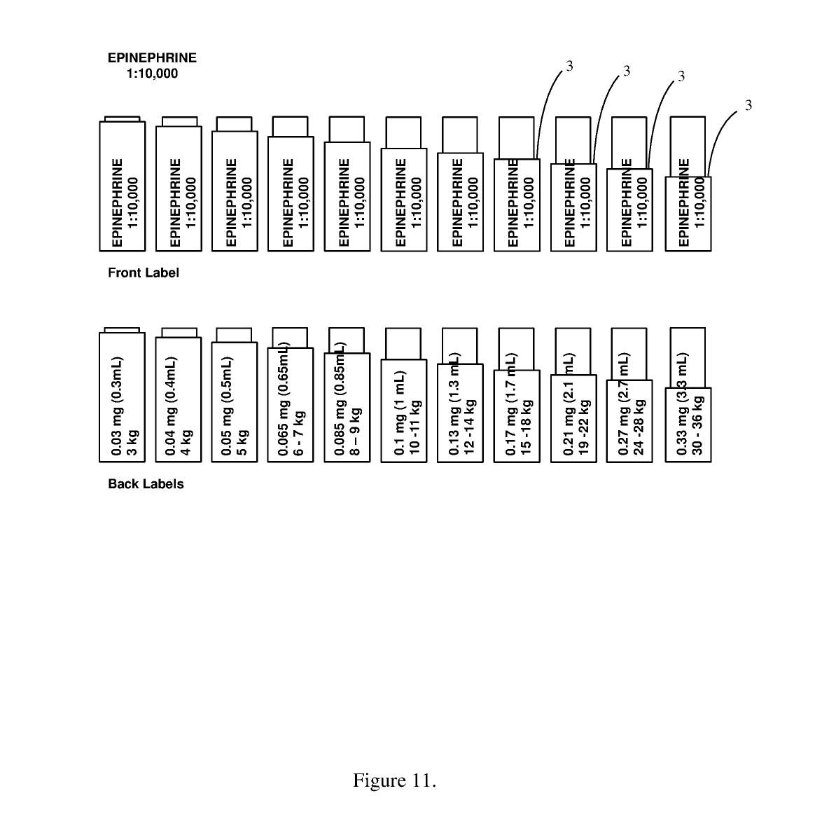

FIG. 11 is a front view of how the invention might appear in an EMT's medication kit, with different color-coded inserts for different medications, with each insert color coded to the appropriate “zone” of the Broselow® tape. The medication here, used by way of example, is Epinephrine, a common emergency medication. The inserts are color coded to the appropriate Broselow® color zone, such that all an EMT needs to do if dealing with a child under emergency circumstances is measure the child with the Broselow® tape and pick out the appropriate insert, then place the insert into the syringe, attach a vial of Epinephrine, and depress the vial of Epinephrine until the lip of the Epinephrine vial hits the blocking ledge of the insert.

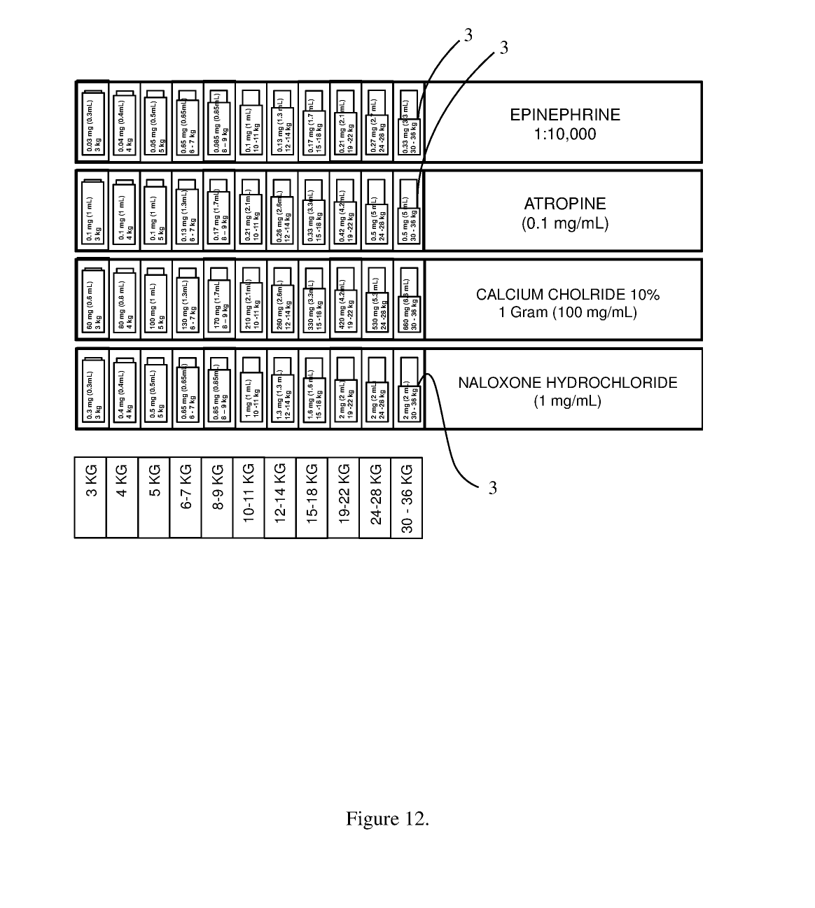

FIG. 12 is a front view of how the invention might appear in an EMT's medication kit, with different color-coded inserts 3 for different medications, with each insert color coded to the appropriate “zone” of the Broselow® tape and the type of medication written on each insert. Note how the various color and size-coded inserts can be organized horizontally, or vertically, by either the medication type or the dosage category. It is also envisioned that other inserts or labels can be attached to the vial to provided various types of information, included but not limited to: name and concentration of the medication, amount based upon dosage weight, amount based on dosage volume, calculated amount based on pediatric patient weight, and color-coding based on Broselow® Pediatric Emergency Tape.

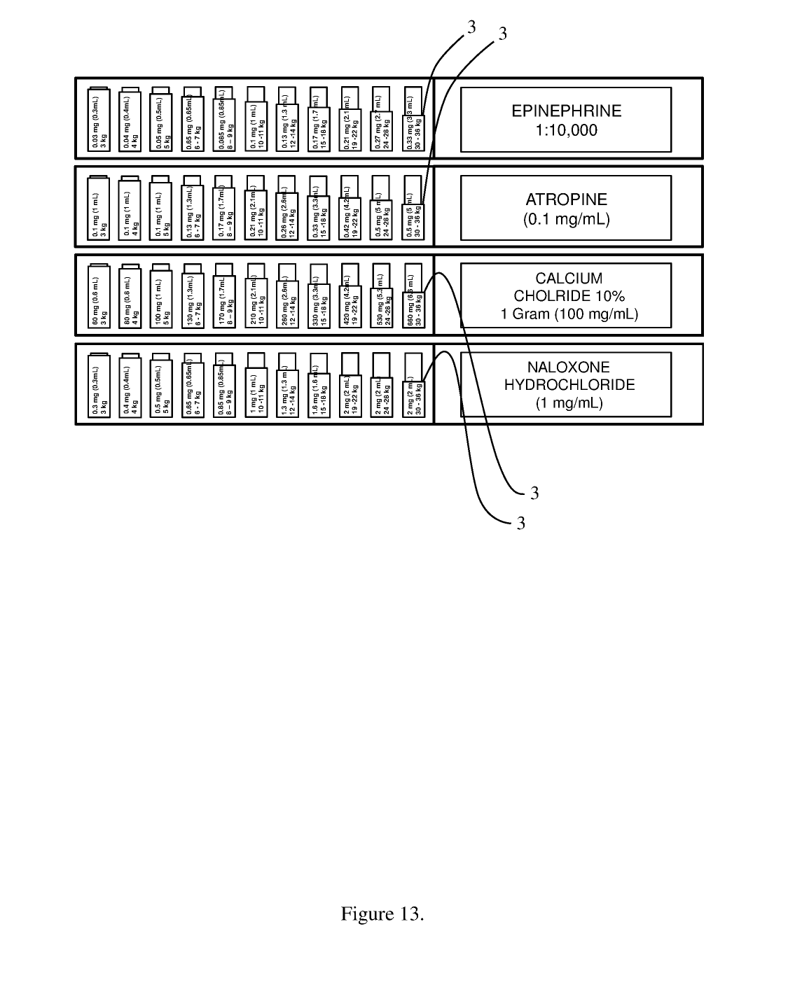

FIG. 13 is a front view of how the invention might appear in another embodiment of an EMT's medication kit, with different color-coded inserts 3 for different medications, with each insert color coded to the appropriate “zone” of the Broselow® tape and the inserts organized by dosage category.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

What I claim is:

1. An insert for regulating the amount of medication to a syringe, comprising, a cylinder with an interior diameter, an insert top portion and an insert bottom portion, where the insert top portion has an insert top portion interior diameter, and the insert bottom portion has an insert bottom portion interior diameter, and where the insert top portion interior diameter is equal to or less than the insert bottom portion interior diameter, and where the insert top portion has an insert top wall thickness, and where the insert bottom portion has an insert bottom wall thickness, and where the insert top wall thickness is less than the insert bottom wall thickness, and where the insert top wall thickness defines an insert top outer diameter and the insert bottom wall thickness defines an insert bottom outer diameter, and where the insert bottom outer diameter is greater than the insert top outer diameter, and where the difference between the insert top outer diameter and the insert bottom outer diameter defines a blocking ledge, where, the insert bottom outer diameter is less than an interior diameter of a syringe, such that the insert can be inserted into the syringe where, the insert top outer diameter is less than an inner diameter of a vial of medication, such that the vial of medication fits over the insert and can be compressed, where the blocking ledge has a diameter greater than the inner diameter of the vial of medication, such that the vial cannot be compressed beyond the blocking ledge, where top edge of the insert can be used by a user of the invention to calibrate the dosage.

2. The insert of claim 1, where, the syringe into which the insert is to be placed is a Hospira Abboject® syringe.

3. The insert of claim 2, where the insert has a color, and where the color corresponds to a “color zone” of a tape that correlates weight range and height range of a child.

4. The insert of claim 3, where the tape that correlates weight range and height range of a child is a Broselow® tape.

5. The insert of claim 4, where the insert has its blocking ledge located at a level that allows for a set dosage of a medication to be delivered to a patient, where the level is set by a location of the blocking ledge on the insert.

6. The insert of claim 5, where the set dosage of medication is equal to a set dosage of the same medication as determined from the color zone of the Broselow® tape for a particular patient.

7. The insert of claim 6, where the insert additionally comprises one or more labels, where the one or more labels provides an information on a proper medication for that insert, a proper concentration for that insert, a proper dosage amount by the volume and weight, and a proper patient weight range for that insert.

8. An insert for regulating the amount of medication to a syringe, comprising a cylinder with an interior diameter and an exterior diameter, where the exterior diameter is greater than or equal to an outer diameter of a vial of medication, such that as the vial of medication is pushed into a syringe, an outer edge of the vial of medication comes into contact with the insert, thereby limiting the amount of medication delivered.

9. The insert of claim 8, where the insert is an insert for regulating the amount of medication to a Hospira Abboject® syringe, where the insert additionally comprises a cylinder with an interior diameter, an insert top portion and an insert bottom portion, where the insert top portion has an insert top portion interior diameter, and the insert bottom portion has an insert bottom portion interior diameter, and where the insert top portion interior diameter is equal or less than the insert bottom portion interior diameter, and where the insert top portion has an insert top wall thickness, and where the insert bottom portion has an insert bottom wall thickness, and where the insert top wall thickness is less than the insert bottom wall thickness, and where the insert top wall thickness defines an insert top outer diameter and the insert bottom wall thickness defines an insert bottom outer diameter, and where the insert bottom outer diameter is greater than the insert top outer diameter, and where the difference between the insert top outer diameter and the insert bottom outer diameter defines a blocking ledge.

10. The insert of claim 9, where, the insert bottom outer diameter is less than an interior diameter of a syringe, such that the insert can be inserted into the syringe.

11. The insert of claim 10, where, the insert top outer diameter is less than an inner diameter of a vial of medication, such that the vial of medication fits over the insert and can be compressed.

12. The insert of claim 11, where the blocking ledge has a diameter greater than the inner diameter of the vial of medication, such that the vial cannot be compressed beyond the blocking ledge.

13. The insert of claim 12, where top edge of the insert can be used by a user of the invention to calibrate the dosage.

14. The insert of claim 13 where the insert additionally comprises one or more labels, where the one or more labels provides an information on a proper medication for that insert, a proper concentration for that insert, a proper dosage amount by the volume and weight, and a proper patient weight range for that insert.

15. A kit of two or more inserts, where each of the two or more inserts comprises a cylinder with an interior diameter, an insert top portion and an insert bottom portion, where the insert top portion has an insert top portion interior diameter, and the insert bottom portion has an insert bottom portion interior diameter, and where the insert top portion interior diameter is equal to the insert bottom portion interior diameter, and where the insert top portion has an insert top wall thickness, and where the insert bottom portion has an insert bottom wall thickness, and where the insert top wall thickness is less than the insert bottom wall thickness, and where the insert top wall thickness defines an insert top outer diameter and the insert bottom wall thickness defines an insert bottom outer diameter, and where the insert bottom outer diameter is greater than the insert top outer diameter, and where the difference between the insert top outer diameter and the insert bottom outer diameter defines a blocking ledge, where, the insert bottom outer diameter is less than an interior diameter of a syringe, such that the insert can be inserted into the syringe where, the insert top outer diameter is less than an inner diameter of a vial of medication, such that the vial of medication fits over the insert and can be compressed, where the blocking ledge has a diameter greater than the inner diameter of the vial of medication, such that the vial cannot be compressed beyond the blocking ledge, where top edge of the insert can be used by a user of the invention to calibrate the dosage.

16. The kit of claim 15, where each insert has the blocking ledge at a different location on the insert.

17. The kit of claim 16, where, the insert bottom outer diameter is less than an interior diameter of a syringe, such that the insert can be inserted into the syringe.

18. The kit of claim 17, where, the insert top outer diameter is less than an inner diameter of a vial of medication, such that the vial of medication fits over the insert and can be compressed.

19. The kit of claim 18, where the blocking ledge has a diameter greater than the inner diameter of the vial of medication, such that the vial cannot be compressed beyond the blocking ledge.

20. The kit of claim 19, where each insert is a different color.

21. The kit of claim 20, where the insert has a color, and where the color corresponds to a “color zone” of a tape that correlates weight range and height range of a child.

22. The kit of claim 21, where the tape that correlates weight range and height range of a child is a Broselow® tape.

23. The kit of claim 22, where the inserts are organized according to the medication.

24. The kit of claim 15, where the inserts are made from plastic.
